SEQUENCE LISTING (12 sequences)

SEQ ID NO: 1 - DNA, 128 bp, Bacillus thuringiensis
  source 1..128: mol_type = genomic DNA
ctagaaccatgaattaaaagaatcacttataaaaaaaatgaagaaataaaaaagacataaagaacaaatatgcataattgcataaagtctggataatttttcatgatatatttaaagaaaaaatgcgg

SEQ ID NO: 2 - DNA, 246 bp, Bacillus thuringiensis
  source 1..246: mol_type = genomic DNA
agcatgtgtatgctttactctttttttacattagtttagacaagccttaataatagtttattaaaatgaaagtgtattcattcattatattcactgtgtataaagttataatgatatgaacatttgcatattttaatttagtgatagaaatttcgtgaaaggtgggatattctagtcataggttaaccggacgacatcataggatcctaacaaaatgtttacaataattcaattataaaatggagg

SEQ ID NO: 3 - DNA, 142 bp, Bacillus thuringiensis
  source 1..142: mol_type = genomic DNA
ttttttcaatatttgttcctcaaaattctacaaaacttgagaaataaattaattgaatttttagtatattaatagtggaaacataatgctaatatgaaactactctttttcaaaaaaatttttattagggggaaggttcatg

SEQ ID NO: 4 - DNA, 122 bp, Bacillus thuringiensis
  source 1..122: mol_type = genomic DNA
gaagtgaagtcaagagagaaaaaaatgaaattatgcatattttttagaaaatttatatttatcaatttatatttctccgaattttatgtattattagagtaatggggtaatgagaatggagg

SEQ ID NO: 5 - DNA, 304 bp, Bacillus thuringiensis
  source 1..304: mol_type = genomic DNA
ctagaccgtcaaaatattactagaattattatactataaaagctataataagtactaggattaattttttgaaaattatacgcaattaaaggtctgattttaagatgatggtagcaatgttaatgtatccctttacgaaaaatttaaaatataaatattttattaaaaattttaacaaaaaaacaaaaaactatattcacaattgttatattatatgtataatgaaaattgtagaaacttggagattattctttcaattctactttttaacaagtgagggtacaggaagtgcaattaggggagg

SEQ ID NO: 6 - DNA, 130 bp, Bacillus thuringiensis
  source 1..130: mol_type = genomic DNA
attccacttttatataaaaagatttttgaatattttttcaacttacccttgtcaaacattgtcaatttatattaaaataacaacatacaaaatatttagtcttttcagaaaagatggagggatagccatg

SEQ ID NO: 7 - DNA, 150 bp, Bacillus thuringiensis
  source 1..150: mol_type = genomic DNA
aattgtgatatattcgtatgctaactatgaaatttttacaaatatattaaaaatattacatgatatgactaaatattgaaaaaatattgaatttttaataaaattcaatttgtaatacatattatttattaggggaggaaatatgggatg

SEQ ID NO: 8 - DNA, 330 bp, Bacillus thuringiensis
  source 1..330: mol_type = genomic DNA
aagcaagactagtaatatttatacgagtgtgagggaacgttagccctcacctctttgtttttcttttttcttatagtattaaatgatttgagtgtgaaaaaagttataaattatcattgtttttttctgaaaagtctaaatgatattgagaaataaaaataactgaaaatattaataaatatgtgttgtgtttatatagggttgttcgttataatgaacataaggtttttaaaaaagaacacatattagctaagctaatatagttttctttacatctcttattgaaaaataagtgataaaaatataaaaaaaagctagggggaattgatt

SEQ ID NO: 9 - DNA, 15 bp, Bacillus thuringiensis
  source 1..15: mol_type = genomic DNA
gaaaggagggatgcc

SEQ ID NO: 10 - DNA, 222 bp, Bacillus thuringiensis
  source 1..222: mol_type = genomic DNA
aaactcaggtttaaatatcgttttcaaatcaattgtccaagagcagcattacaaatagataagtaatttgttgtaatgaaaaacggacatcacctccattgaaacggagtgatgtccgttttactatgttattttctagtaatacatatgtatagagcaacttaatcaagcagagatattttcacctatcgatgaaaatatctctgctttttctttttttat

SEQ ID NO: 11 - DNA, 300 bp, synthetic construct
  misc_feature 1..300: note = Squence contenant 2 domaines LysM tels que dcrits par Shao etal., 2009, Microb Cell Fact 8, 48. doi:10.1186/1475-2859-8-48.
  source 1..300: mol_type = other DNA
atgattcaaattgtaacggttcgtagcggtgatagcgtatatagcttggcatcaaaatatggatcaacacctgacgaaatagtaaaagacaatggactaaatcccgctgaaacgctcgttgttggtcaggcacttatcgttaatacgaaaggaaataattattatgtacagcctggtgacagcctctatcggatttctcaaacatataatgtccccctcgctagtttagctaaagttaataatttatctttaaaatctattctccatgtcggacaacaattatatgtaccaaaaggcaca

SEQ ID NO: 12 - DNA, 546 bp, synthetic construct
  misc_feature 1..546: note = Squence contenant 3 domaines SLH tels que dcrits par Fedhila etal., 2006, Mol. Microbiol. 62: 339-355. doi:10.1111/j.1365-2958.2006.05362.x
  source 1..546: mol_type = other DNA
gaagatgagaaaacagaagtggtagaatttaaagatgtaccaaagggacattggtcagaagaagcaattaattacttagcgaaagaaaaattatttataggctatggaaatggtgaatttggatttggtgataacattactcgtggacaagtagcacttctaatacaaagatatttaaaattagaaaataatctagaacaaaaaacggcatttacagatacgaaaggaaatatgtatgaaacggctattgatgcagtggttcaagctggaattatgacaggctatggaaatggtatgttccgtccggatggagtattaactcgatatgaaatgtcagtagtactacaaagagtatttcagttaaaagaaaatgaaaatagtgcagagaattttaaagatgtaccaaatggccattgggcgaaaggatatgtgaaagctttagtggataataaaatatcaaaaggcgacggggaagggaattttttaggagataatttcgtaacacgtgaacaatacgcacagtttttgtataatgcaataaagaaa

(19) United States
(12) Patent Application Publication
(10) Pub. No.: US 2025/0297209 A1
(43) Pub. Date: Sep. 25, 2025

(54) PRODUCTION OF PROTEINS OF INTEREST IN A NON-SPORULATING BACTERIAL STRAIN

(71) Applicants: INSTITUT NATIONAL DE RECHERCHE POUR L'AGRICULTURE, L'ALIMENTATION ET L'ENVIRONNEMENT, PARIS (FR); INSTITUT NATIONAL DES SCIENCES ET INDUSTRIES DU VIVANT ET DE L'ENVIRONNEMENT, PALAISEAU (FR); UNIVERSITE PARIS-SACLAY, GIF-SUR-YVETTE (FR)

(72) Inventors: Leyla SLAMTI, PARIS (FR); Didier LERECLUS, VERSAILLES (FR); Michel GOHAR, TERCE (FR)

(21) Appl. No.: 18/862,275

(22) PCT Filed: Apr. 26, 2023
(86) PCT No.: PCT/EP2023/061003

§ 371 Date: Nov. 1, 2024

(30) Foreign Application Priority Data
May 2, 2022 (FR) FR2204132

(51) Int. Cl. C12N 1/20; C07K 14/32; C12R 1/07

(52) U.S. Cl. CPC C12N 1/20; C07K 14/32; C12R 2001/075

(57) ABSTRACT

The present disclosure relates to a system for the production of proteins of interest composed of a non-sporulating bacterial strain of the genus Bacillus transformed with a plasmid containing an expression cassette of a protein of interest under the control of a strong promoter active in the stationary phase. The proteins of interest thus produced are present in a bacterial sacculus or anchored to the surface of the bacterium.

### CROSS-REFERENCE TO RELATED APPLICATION(S)

This present application is a national stage application of International Patent Application No. PCT/EP2023/061003, filed Apr. 26, 2023, which claims priority to French Patent Application No. 2204132, filed May 2, 2022, the disclosures of which are hereby incorporated by reference in their entireties.

### TECHNICAL FIELD

The present disclosure relates to a novel system for the production of proteins of interest composed of a non-sporulating bacterial strain of the genus Bacillus transformed with a plasmid containing an expression cassette of a protein of interest under the control of a strong promoter active in the stationary phase. The proteins of interest thus produced are present in a bacterial sacculus consisting of the bacterial membrane or anchored to the surface of the bacterium.

### BACKGROUND

The use of recombinant bacteria to produce proteins of interest, such as insulin and growth hormone, has been known for a long time and has been widely implemented, but it has also shown its limitations. Indeed, the bacteria are unable to produce proteins with a complex structure such as antibodies or blood coagulation factors. To be stable and active in vivo and therefore effective in humans, these proteins must undergo multiple post-translational modifications.

It also happens that heterologous proteins of interest are toxic to the bacterial cell producing them. Vincent Ecochard et al., in “Techniques et stratégies en biologie moléculaire,” professional master's degree, October 2011, http://www.m2p-egpr.ups-tlse.fr/Documents % 20archives/Cours/Cours %20partie %202.pdf, detail the solutions that can be provided to produce such proteins in a bacterial system. However, it is not easy to identify the best solution in relation to the protein to be produced. The Authors indeed conclude that the last solution and the best recourse is to use an in vitro translation system.

Rosano et al., in “Recombinant protein expression in Escherichia coli: advances and challenges,” Front. Microbiol., 17 Apr. 2014, https://doi.org/10.3389/fmich.2014.00172, indicate that only a few strains of E. coli are capable of producing toxic proteins, but the level of production of these proteins is low. A solution could be provided by secreting the protein outside the bacterial cell or into the periplasm, using different promoters.

A bacterial system for the production of proteins of interest that is simple to implement and with a high yield is therefore necessary, in particular for the production of proteins of interest that may be toxic.

Bacillus thuringiensis (Bt) is a Gram-positive sporulating bacterium that produces large amounts of insecticidal proteins (Cry and Cyt proteins). When certain Bt sporulation genes are no longer expressed, such as spo0A, sigE or sigF, or when their product is no longer functional following a mutation, the bacterium is unable to enter into sporulation and remains blocked in the stationary phase. A consequence of the inactivation of the sporulation genes is the cessation of the bacterial multiplication, the bacteria are therefore non-viable.

The inventors have shown that a non-sporulating Bt strain leads to the formation of bacterial sacculus. Surprisingly, they have also shown that, in such non-sporulating strains, the production of proteins of interest can be obtained by placing the gene of interest under the control of a promoter specifically activated during the stationary phase. Thus, the protein of interest is produced during the stationary phase and remains encapsulated in the bacterial sacculus. The inventors have also shown that these proteins are produced in large quantities and that their localization in the bacterial sacculus protects them from degradation and greatly facilitates their recovery and their purification. According to a particular embodiment, it is also possible to express the proteins of interest so that they anchor themselves to the surface of the bacterial cells.

This novel system therefore makes it possible to produce proteins of interest, in particular proteins that are unstable or toxic to the producing bacterial cell, the latter being non-sporulating and non-viable.

Thus, the present disclosure relates to a non-sporulating bacterial strain of the genus Bacillus which contains a recombinant plasmid comprising an expression cassette composed of:
a strong promoter active in the stationary phase and regulated by a regulator chosen from CodY, AbrB, SinR, PlcR and NprR; and
the sequence of a gene encoding a protein of interest.

Preferably, the bacterial strain is chosen from the strains of Bacillus thuringiensis, Bacillus cereus, Bacillus weihenstephanensis, and more preferably it is a strain of Bacillus thuringiensis. The strains Bacillus subtilis, Bacillus megaterium, Bacillus brevis, can also be used provided that they are previously transformed to express the papR and plcR genes activating respectively the PpapR and PplcR promoters and/or to express the npnR and nprX genes activating the PnprA promoter.

Preferably, the used bacterial strains are the Bt kurstaki HD-73 or Bt 407 strains. According to the disclosure, the bacterial strain is a non-sporulating strain. Indeed, the non-sporulating strains are advantageous in that they do not have cell lysis, thus, the produced proteins of interest are preserved in the producing bacterium and protected from degradation by the extracellular proteases.

In order to suppress the sporulation activity of a strain of the genus Bacillus, it is possible to inactivate any gene essential for sporulation such as the genes involved in the expression of the Spo0A transcriptional regulator responsible for the initiation of sporulation or in the expression of the SigE, SigF, SigH and SigK sporulation sigma factors; preferably, the inactivated gene is Spo0A or sigE. The inactivation of these genes can be obtained by interruption or modification of the coding sequence, or by deletion of all or part of the gene.

The deletion is obtained by double crossing-over between the adjacent regions located upstream and downstream of the gene, using plasmids whose replication is thermosensitive, for example the plasmids pRN5101 (Lereclus et al., Expansion of insecticidal host range of Bacillus thuringiensis by in vivo genetic recombination. Biotechnology (NY) 10: 418-421, 1992) or pMAD (Arnaud et al., New vector for efficient allelic replacement in naturally nontransformable, low-GC-content, gram-positive bacteria. Appl Environ Microbiol 70:6887-6891, 2004), and using the protocols described in these articles. Deletion of the Spo0A gene (designated ASpo0A) has a very early effect, as soon as the bacteria enter into the stationary phase, notably preventing the bacteria from engaging in the sporulation process (Lereclus et al., Overproduction of encapsulated insecticidal crystal proteins in a Bacillus thuringiensis Spo0A mutant. Biotechnology (NY) 13:67-71, 1995). Deletion of the sigE gene (designated ΔsigE) has a later effect, blocking the progression of the sporulation process (Bravo et al., Analysis of crylAa expression in sigh and sigK mutants of Bacillus thuringiensis. Mol Gen Genet 250:734-741, 1996). In both cases, the bacteria no longer multiply, die and contain almost only the protein of interest.

Preferably, the used strain is Bt kurstaki HD-73 ΔSpo0A or Bt 407 ΔsigE.

The recombinant plasmids that can be used according to the disclosure conventionally comprise an origin of replication and at least one selection system consisting of one or more genes allowing the selection of the transformed bacteria, for example antibiotic resistance genes. Any plasmid, preferably with a high copy number, adapted to the used bacterial host can be implemented.

Suitable plasmids according to the disclosure are plasmids allowing expression in bacteria of the genus Bacillus.

Preferably, the used plasmids are those having high segregational and/or structural stability.

The notion of segregational stability means that the plasmid is not lost over generations; in other words, it is stably maintained in the bacterial cell and transmitted to daughter cells.

The segregational stability of the plasmid pHT1030 has been demonstrated (Lereclus et al., 1992. spbA locus ensures the segregational stability of pHT1030, a novel type of Gram-positive replicon. Mol. Microbiol. 7:35-46). This stability is due to the presence of the spbA gene which is also present in the plasmids derived from pHT1030, such as pHT3101, pHT304, pHT315 and pHT370 (Arantes et al., 1991. Construction of cloning vectors for Bacillus thuringiensis. Gene 108:115-119). The segregational stability of the plasmid pBC16 and its derivatives was also determined (Lereclus et al., 1992. spbA locus ensures the segregational stability of pHT1030, a novel type of Gram-positive replicon. Mol. Microbiol. 7:35-46).

The structural stability is defined as non-intramolecular recombination of the plasmid. The structural stability of plasmid pHT1030 and its derivatives is demonstrated by the fact that they can carry large exogenous DNA fragments (>10 kb) without undergoing molecular rearrangements (Lereclus et al., 1989. Transformation and expression of a cloned delta-endotoxin gene in Bacillus thuringiensis. FEMS Microbiol. Lett. 60:211-217).

Preferably, these are high copy number plasmids, in particular derived from plasmid pHT1030, such as pH3101, pHT304, pHT315 and pHT370, preferably pH315, or derived from plasmid pBC16, pE194 or pC194 or low copy number plasmids such as pHT73, resident plasmid of the Bt kurstaki HD-73 or pBMB299 strain, resident plasmid of the Bt kurstaki HD1 strain.

According to the disclosure, the expression cassette inserted into said plasmid contains at least one strong promoter and the sequence of a gene encoding a protein of interest.

A strong promoter is a promoter that allows a strong transcription of the gene(s) it controls. The strong promoter can come from the host cell used for the expression of the protein of interest; it can also be an exogenous promoter. The strong promoters are preferentially promoters that are activated at the beginning of the stationary phase and that remain functional for a large part of this physiological state. Commonly, the expression of the promoters is evaluated with the activity of R-galactosidase (AS, in units/mg of protein) according to the calculation described in Perchat et al. (“A cell-cell communication System regulates protease production during sporulation in bacteria of the Bacillus cereus group,” Molecular Biology, Vol. 82, 3, p. 619-633, 2011): AS=(A₄₂₀×1500000)/(T×V×C).
A₄₂₀: absorbance at 420 nm
T: reaction time in min
V: volume of the crude extract in μL
C: protein concentration in μg/mL

Using this formula, a promoter is considered highly expressed when the AS is greater than 500 U/mg prot.

According to a first embodiment of the disclosure, the strong promoter is regulated by a regulator chosen from PlcR and NprR. The strong promoters are preferentially chosen from PpapR (SEQ. ID. NO: 1), PplcB (SEQ. ID. NO: 2), PprA (SEQ. ID. NO: 3) and PnprR (SEQ. ID. NO: 4).

According to a second embodiment of the disclosure, the strong promoter is regulated by a regulator chosen from CodY, AbrB and SinR and even more preferentially the strong promoter is chosen from PoppA (SEQ. ID. NO. 5), PnppC (SEQ. ID. NO. 6), PinhA1 (SEQ. ID. NO. 7) and Pco/Y(SEQ. ID. NO. 8).

Preferably, the regulator is CodY and the strong promoter is PoppA or PnppC.

The expression cassette according to the disclosure can further comprise:
a stabilizing sequence of mRNA positioned downstream of the promoter and upstream of the sequence of the gene encoding the heterologous protein; preferably, it is STAB-SD of sequence SEQ ID NO. 9; preferably, the STAB-SD sequence is downstream of the transcription+1 and at a position comprised between approximately 100 and 500 nucleotides upstream of the ribosome binding site (RBS), preferentially between 100 and 300 nucleotides and more preferentially between 100 and 150 nucleotides; and/or
a terminator sequence of the cry1Ac gene, designated Tcry1Ac, for example a sequence having at least 90% identity with SEQ ID NO. 10, preferably, it is SEQ ID NO. 10; this sequence is cloned downstream of the gene encoding the protein of interest.

The choice of these sequences should not, however, be considered as limiting since it is within the reach of those skilled in the art to substitute these sequences with sequences with equivalent functions.

The terms “protein of interest” mean an endogenous protein or a protein that is not naturally expressed by the bacterial strain according to the disclosure, also referred to as a heterologous protein. Preferably, the protein of interest is a cytotoxic protein naturally expressed by a strain of the genus Bacillus or a protein of industrial interest such as enzymes, such as proteases, lipases, amylases; hormones; antigens, for example, usable as immunogens, peptides or proteins for therapeutic use; the protein of interest can thus find application in the field of crop protection, vector control, commercial production of enzymes and the pharmaceutical industry, in particular for the production of vaccines.

The expression cassette according to the disclosure leads to the expression and then the accumulation of proteins of interest in bacterial sacculus or to their anchoring to the surface of the bacteria. Its use is particularly suitable for the production of proteins that are unstable or toxic to the producing strain.

The construction of the expression cassette according to the disclosure and its incorporation into a plasmid are carried out by the molecular biology techniques well known to those skilled in the art as illustrated in the experimental part.

The plasmid according to the disclosure can be introduced into the host bacterium according to the techniques known to those skilled in the art; in particular, the transformation of the host bacterium can be carried out by electroporation (Lereclus et al., 1989) or by heterogramic conjugation (Tieu-Cuot et al., 1987). The expression cassette containing the gene of interest can also be introduced onto the bacterial chromosome or onto a resident plasmid by homologous recombination (Lereclus et al., 1992).

### SUMMARY

The present disclosure also relates to a method for producing a protein of interest comprising the steps of:
preparing the bacterial strain according to the disclosure;
culturing said bacterial strain in the stationary phase; and
optionally, purifying said protein of interest.

The culture of the bacterial strain is carried out on a culture medium containing at least one source of nitrogen and glucose in appropriate concentrations at a temperature comprised preferably between 25 and 35° C., preferably the temperature is in the range of 30° C.; for example, the culture medium is LB medium.

The purification of the protein of interest can be carried out by centrifugation; in addition, exclusion chromatography, ion exchange chromatography or even affinity chromatography methods can be implemented.

According to a particular embodiment, a gene encoding for an export protein domain (such as a signal peptide) or anchoring (such as LysM (SEQ. ID NO. 11), SLH (SEQ ID NO. 12) or LPXTG (Navarre et al., Microbiol Mol Biol Rev 63(1): 174-229.DOI: 10.1128, 1999)) of the proteins on the surface of bacteria is cloned in cis of the gene encoding the protein of interest.

Thus, the expression cassette according to the disclosure can comprise:
(i) a strong promoter active in the stationary phase chosen from PpapR, PplcB, PnprA, PnprR, PoppA, PnppC, PinhA1 and PcalY and preferentially chosen from PoppA and PnppC;
(ii) optionally, the sequence of a gene encoding for an export or anchor protein;
(iii) the sequence of a gene encoding a protein of interest;
and optionally a stabilizing sequence of mRNA, preferably STAB-SD, and/or the terminator sequence of the cry1Ac, Tcry1Ac gene;
or
(i) a strong promoter active in the stationary phase chosen from PpapR, PplcB, PnprA, PnprR, PoppA, PnppC, PinhA1 and PcalY and preferentially chosen from PoppA and PnppC;
(ii) optionally, the sequence of a gene encoding for an export or anchor protein;
(iii) the sequence of a gene encoding a protein of interest;
and a stabilizing sequence of mRNA, preferably STAB-SD;
or
(i) a strong promoter active in the stationary phase chosen from PpapR, PplcB, PnprA, PnprR, PoppA, PnppC, PinhA1 and PcalY and preferentially chosen from PoppA and PnppC;
(ii) optionally, the sequence of a gene encoding for an export or anchor protein;
(iii) the sequence of a gene encoding a protein of interest;
and the terminator sequence of the cry1Ac, Tcry1Ac gene;
or even
(i) a strong promoter active in the stationary phase chosen from PpapR, PplcB, PnprA, PnprR, PoppA, PnppC, PinhA1 and PcalY and preferentially chosen from PoppA and PnppC;
(ii) optionally, the sequence of a gene encoding for an export or anchor protein;
(iii) the sequence of a gene encoding a protein of interest;
and a stabilizing sequence of mRNA, preferably STAB-SD, and the terminator sequence of the cry1Ac, Tcry1Ac gene.

According to particular embodiments, the strain used is a bacterium of the genus Bacillus ΔSpo0A, preferably Bt ΔSpo0A, more preferably Bt HD73 ΔSpo0A, and the expression cassette comprises:
(i) a strong promoter active in the stationary phase chosen from PpapR, PplcB, PoppA, PnprR, PnppC and PnprA and preferentially chosen from PoppA and PnppC;
(ii) optionally, the sequence of a gene encoding for an export or anchor protein;
(iii) the sequence of a gene encoding a protein of interest;
and optionally a stabilizing sequence of mRNA, preferably STAB-SD, and/or the terminator sequence of the cry1Ac, Tcry1Ac gene;
or
(i) a strong promoter active in the stationary phase chosen from PpapR, PpIcB, PoppA, PnprR, PnppC and PnprA and preferentially chosen from PoppA and PnppC;
(ii) optionally, the sequence of a gene encoding for an export or anchor protein;
(iii) the sequence of a gene encoding a protein of interest;
and a stabilizing sequence of mRNA, preferably STAB-SD;
or
(i) a strong promoter active in the stationary phase chosen from PpapR, PpIcB, PoppA, PnprR, PnppC and PnprA and preferentially chosen from PoppA and PnppC;
(ii) optionally, the sequence of a gene encoding for an export or anchor protein;
(iii) the sequence of a gene encoding a protein of interest;
and the terminator sequence of the cry1Ac, Tcry1Ac gene;
or even
(i) a strong promoter active in the stationary phase chosen from PpapR, PpIcB, PoppA, PnprR, PnppC and PnprA and preferentially chosen from PoppA and PnppC;
(ii) optionally, the sequence of a gene encoding for an export or anchor protein;
(iii) the sequence of a gene encoding a protein of interest;
and a stabilizing sequence of mRNA, preferably STAB-SD, and/or the terminator sequence of the cry1Ac, Tcry1Ac gene.

According to other particular embodiments, the strain used is a bacterium of the genus Bacillus ΔsigE, preferably Bt ΔsigE, even more preferably Bt 407 ΔsigE and the expression cassette comprises:
(i) a strong promoter active in the stationary phase chosen from PcalY and PinhA1;
(ii) optionally, the sequence of a gene encoding for an export or anchor protein;
(iii) the sequence of a gene encoding a protein of interest;
and optionally a stabilizing sequence of mRNA, preferably STAB-SD, and/or the terminator sequence of the cry1Ac, Tcry1Ac gene;
or
(i) a strong promoter active in the stationary phase chosen from PcalY and PinhA1;
(ii) optionally, the sequence of a gene encoding for an export or anchor protein;
(iii) the sequence of a gene encoding a protein of interest, preferably STAB-SD;
and a stabilizing sequence of mRNA;
or
(i) a strong promoter active in the stationary phase chosen from PcalY and PinhA1;
(ii) optionally, the sequence of a gene encoding for an export or anchor protein;
(iii) the sequence of a gene encoding a protein of interest;
and the terminator sequence of the cry1Ac, Tcry1Ac gene;
or even
(i) a strong promoter active in the stationary phase chosen from PcalY and PinhA1;
(ii) optionally, the sequence of a gene encoding for an export or anchor protein;
(iii) the sequence of a gene encoding a protein of interest;
and a stabilizing sequence of mRNA, preferably STAB-SD, and/or the terminator sequence of the cry1Ac, Tcry1Ac gene;
According to a particular embodiment, the produced protein of interest is an insecticidal protein of B. thuringiensis.

It is known that such proteins can be used as biopesticides in preparations conventionally containing the free insecticidal protein in crystal form and bacterial spores, to combat crop pests as well as disease vectors such as mosquitoes. In particular, these may be proteins of the Cry family (crystal proteins) or proteins of the Cyt family (cytolytic insecticidal toxins) or Vip3 proteins (toxins active against lepidopteran insects) and more preferentially proteins of the Cry family.

After their expression, they are protected from degradation by the bacterial membrane which constitutes the envelope of the bacterial sacculus. In addition, the non-sporulation of the bacteria gives the disclosure the advantage of not disseminating the spores in the environment.

According to another embodiment, the produced protein of interest can be an enzyme of industrial (proteases, lipases, amylases, . . . ) or medical interest. The encapsulation of this protein in the bacterial sacculus facilitates its recovery and purification, and therefore reduces the production costs.

According to a third embodiment, the produced protein of interest can be a whole protein or a protein fragment which can serve as an antigen, for example proteins from microorganisms (viruses, bacteria, fungi) or parasites.

In this embodiment, it can be advantageous for the protein or its fragment to be anchored to the surface of the bacterial sacculus. This embodiment is of marked interest for the preparation of vaccines.

Thus, the present disclosure provides a bacterial platform that can be advantageously exploited for the production of proteins of interest in multiple fields such as crop protection, vector control, commercial production of enzymes and the pharmaceutical industry. In addition, this technology has a low cost and excellent yield allowing mass production.

### BRIEF DESCRIPTION OF THE DRAWINGS

FIG. 1 A, HD73 wt strain; B, HD73 ΔSpo0A strain.

FIG. 2 407 ΔsigE strain

FIG. 3 Measurement of the β-galactosidase activity in the HD73 wt bacteria carrying the transcriptional fusion PplcB-STAB-SD-lacZ (black curve) or the transcriptional fusion PplcB-lacZ (gray curve).

FIG. 4 Measurement of the β-galactosidase activity in HD73 Δspo0A bacteria carrying the transcriptional fusion PpapR-STAB-SD-lacZ (gray curve) or the transcriptional fusion PoppA-STAB-SD-lacZ (black curve).

FIG. 5 Measurement of the β-galactosidase activity in 407 ΔsigE bacteria carrying the transcriptional fusion PcalY-STAB-SD-lacZ-TermCry1Ac (black curve) or the transcriptional fusion PcalY-STAB-SD lacZ (gray curve).

FIG. 6 Measurement of the fluorescence at different culture times, in HD73 wt (gray) and HD73 Δspo0A (black) bacteria carrying the transcriptional fusion PpapR-STAB-SD-gfp-TermCry1Ac.

FIG. 7 Production of toxins of interest under the control of the promoters PoppA and PpapR associated with the stabilizing elements STAB-SD and TermCry1Ac. Microscopic observation of a strain HD73 Δspo0A transformed with a plasmid carrying the transcriptional fusions PoppA-STAB-SD-tox1-TermCry1Ac and PpapR-STAB-SD-tox2-TermCry1Ac (A) or of a strain HD73 Δspo0A not containing the plasmid described above (B). SDS-polyacrylamide gel electrophoresis of the strain producing the toxins of interest (C). M, molecular weight marker.

### DETAILED DESCRIPTION

### Examples

### 1. Effect of Creating a Non-Sporulating Phenotype on the Formation of Bacterial Sacculus

The asporulating strains correspond to the Bt HD73 wt strain in which the Spo0A gene was deleted (Bt HD73 ΔSpo0A) or to the Bt 407 wt strain in which the sigh gene was deleted (Bt 407 AsigE). For these two strains, the Spo0A and sigE genes were deleted by double crossing over using the plasmid pMAD and the protocol described by Arnaud et al. (Arnaud et al., 2004)

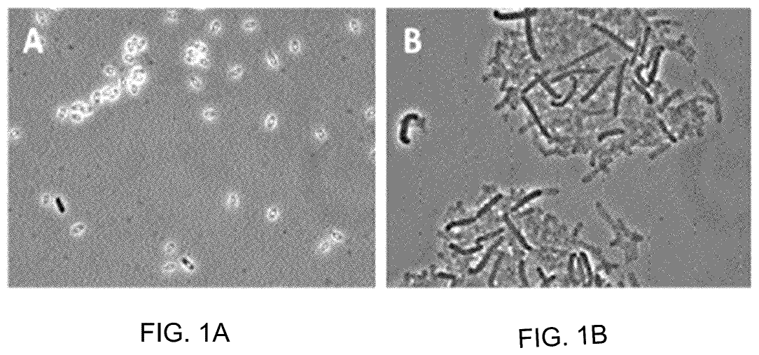

The culture corresponding to the Bt HD73 wt strain consists almost exclusively of spores (FIG. 1A). In contrast, no spores are visible in the case of the Bt HD73 ΔSpo0A mutant (FIG. 1B).

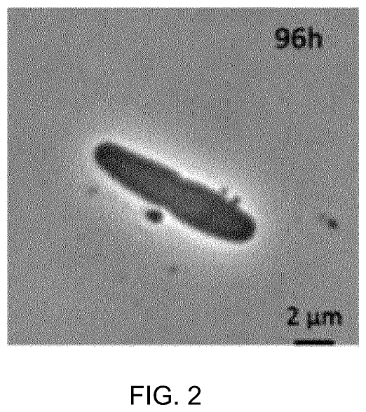

The Bt HD73 Δspo0A mutant shows the formation of bacterial sacculus (light gray in FIG. 1B). Similarly, unlike the Bt 407 wt strain, the Bt 407 ΔsigE strain does not form any spores and is composed of bacterial sacculus (FIG. 2). The Bt HD73 wt and Δspo0A strains were cultured in HCT YEG liquid medium at 30° C. for 72 h before being examined under the microscope. This medium is composed of HCT medium (0.7% casein hydrolysate, 0.5% tryptone, 0.68% KH₂PO₄, 0.012% MgSO₄ 7H₂O, 0.00022% MnSO₄ 4H₂O, 0.0014% ZnSO₄ 7H₂O, 0.008% ferric ammonium citrate, 0.018% CaCl₂) 4H₂O at pH 7.2) supplemented with 0.3% glucose and 0.05% yeast extract.

The Bt 407 ΔsigE strain was cultured in HCT YEG liquid medium at room temperature for 96 h before being examined under the microscope.

### 2. Effect of the Stabilizing Sequence STAB-SD on the Promoter Activity of a Stationary Phase Gene

The inventors added the RNA stabilizing sequence STAB-SD between the PplcB promoter and the lacZ reporter gene on the plasmid pHT304-18Z-PplcB to generate the plasmid pHT304-18-PpIcB-STAB-SD-lacZ. The RNA stabilizing sequence STAB-SD was first cloned between the XbaI and BamHI restriction sites of the vector pHT304-18. Then, the PplcB promoter was cloned upstream between the PstI and XbaI restriction sites.

The Bt HD73 wt strain was transformed with the plasmids pHT304-18Z-PplcB and pHT304-18-PpIcB-STAB-SD-lacZ and then cultured in LB medium at 30° C.

LB (Luria Bertani) culture medium is a complex medium conventionally used for the cultivation of the bacterial strains, it is composed of tryptone, 10 g/L; yeast extract, 5 g/L; NaCI, 10 g/L. The components are solubilized in 800 mL of demineralized water, the pH is then adjusted to 7.0 and the volume is made up to 1 L. The medium is sterilized by autoclaving at 121° C. for 15 minutes.

t0 corresponds to the beginning of the transition phase between the exponential phase and the stationary growth phase.

Thus, the activity of PplcB, promoter active in the stationary phase, was compared in the presence and absence of STAB-SD.

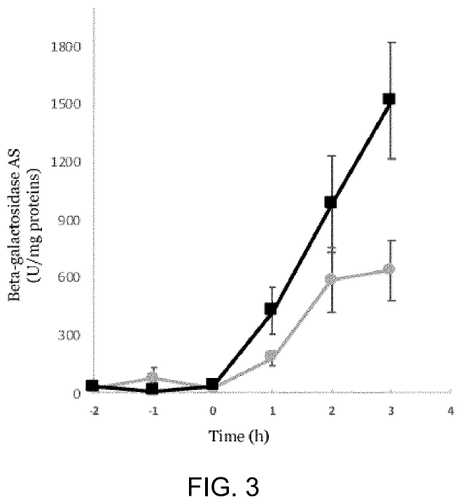

The results in FIG. 3 show that the PplcB promoter is active in both vector constructs: in the presence and absence of STAB-SD. The presence of STAB-SD increases the expression of the β-galactosidase.

### 3. Activity of Stationary Phase Promoters in the ΔSpo0A Mutant

DNA fragments corresponding to the sequences of the promoters of PoppA or PpapR stationary phase genes followed by the sequence STAB-SD were synthesized and cloned into the vector pHT315. The lacZ reporter gene was cloned downstream of these sequences. The vectors were introduced into Bt HD73 ΔSpo0A cells.

t0 corresponds to the beginning of the transition phase between the exponential phase and the stationary growth phase.

The bacterial strains were cultured in HCT YEG medium at 30° C.

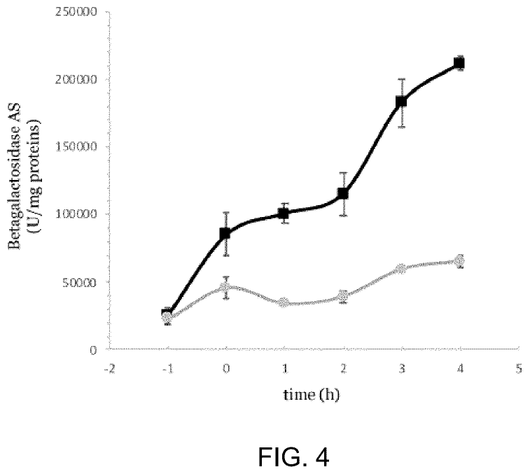

The β-galactosidase activity of these strains was measured during their growth and shows a strong activity of these promoters at the times indicated in FIG. 4, and a greater activity for the PoppA promoter from to.

### 4. Effect of the Stabilizing Sequence TermCry1Ac on the Expression of a Stationary Phase Gene

The sequence corresponding to the terminator of the cry1Ac gene was cloned downstream of the transcriptional fusion between the promoter of a gene expressed in the stationary-phase (PcalY) and the lacZ reporter gene on the plasmid pHT304.18Z to generate the plasmid pPcalY-STAB-SD-lacZ-TermCry1Ac. This vector was introduced into Bt 407 ΔsigE cells.

t0 corresponds to the beginning of the transition phase between the exponential phase and the stationary growth phase.

The bacterial strains were cultured in LB medium at 30° C.

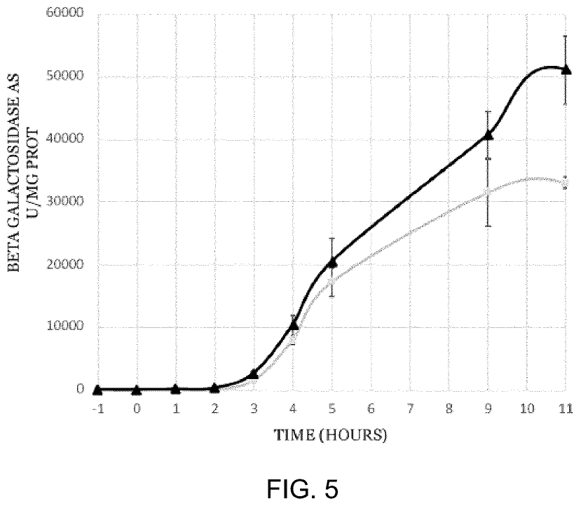

The β-galactosidase activity of this strain was measured and compared to that of a strain carrying the same vector except for the sequence TermCry1Ac. The results show that both plasmids allow the expression of the β-galactosidase, the fusion carrying the sequence TermCry1Ac leads to a stronger expression than the one not carrying it (FIG. 5).

### 5. Effect of the Association of the Genetic Elements on the Expression of a Reporter Gene

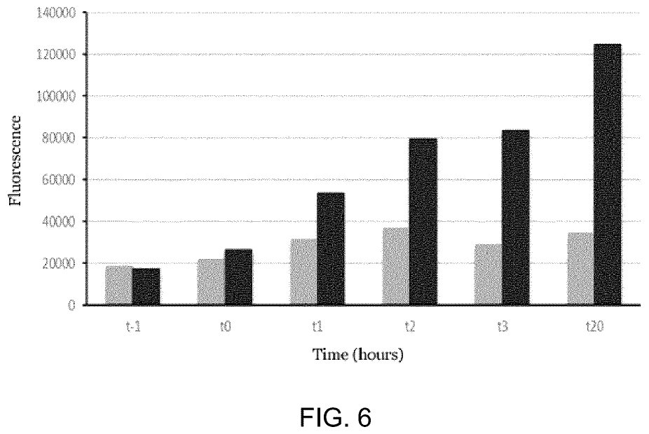

The Bt HD73 wt and Bt HD73 ΔSpo0A strains were transformed with the plasmid pHT315 carrying the transcriptional fusion PpapR-STAB-SD-gfp-TermCry1Ac in order to measure the activity of this transcriptional fusion in the ΔSpo0A genetic background and to compare it to the activity of the wt strain (FIG. 6).

The plasmid was constructed as follows. The DNA fragment corresponding to the sequence of the PpapR promoter followed by the sequences STAB-SD and TermCry1Ac was synthesized and cloned into the vector pHT315. The gfp reporter gene was cloned downstream of this sequence.

t0 corresponds to the beginning of the transition phase between the exponential phase and the stationary growth phase.

The bacterial strains were cultured in HCT YEG medium at 30° C.

The results show that the association of the different genetic elements (plasmid, PpapR promoter, sequence STAB-SD and sequence TermCry1Ac) is functional in the HD73 ΔSpo0A strain. In addition, the measurement of the fluorescence produced by the HD73 ΔSpo0A and HD73 wt strains shows that the expression of the gfp gene is stronger in the ΔSpo0A genetic background than in the wild background.

### 6. Effect of the Association of the Genetic Elements on the Production of Insecticidal Proteins

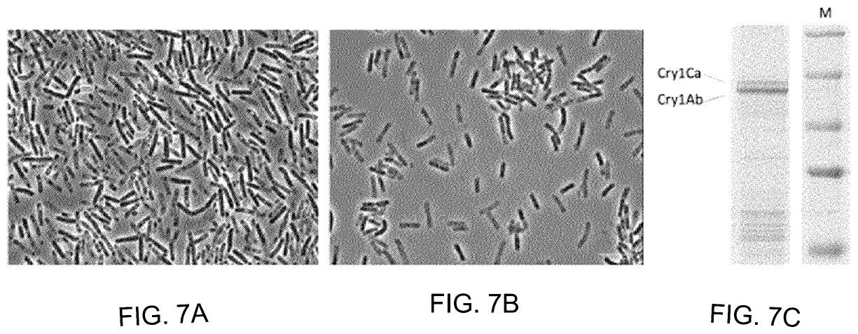

A HD73 ΔkSpo0A strain was transformed with a plasmid carrying the transcriptional fusions PoppA-STAB-SD-cry1Ab-TermCry1Ac and PpapR-STAB-SD-cry1Ca-TermCry1Ac. The production of the toxins of interest Cry1Ab and Cry1Ca corresponding to the cry1Ab and cry1Ca genes, respectively, was verified by phase contrast microscopy and by SDS-polyacrylamide gel electrophoresis (FIG. 7). The bacteria were cultured in PEP YEGx medium at 30° C. and harvested 48 h post-inoculation. This medium is composed of 0.7% casein hydrolyzate, 0.5% peptone, 0.68% KH₂PO₄, 0.012% MgSO₄7H₂O, 0.00022% MnSO₄4H₂O, 0.0014% ZnSO₄7H₂O, 0.008% ferric ammonium citrate, 0.018% CaCl₂) 4H₂O, at pH 7.2 supplemented with 0.3% glucidex and 0.05% yeast extract.

The results show the production of the toxins in crystal form in HD73 ΔSpo0A bacterial sacculus carrying PoppA-STAB-SD-cry1Ab-TermCry1Ac and PpapR-STAB-SD-cry1Ca-TermCry1Ac (FIG. 7A) while a HD73 ΔSpo0A strain not containing the plasmid described above has bacterial sacculus without crystals (FIG. 7 B). Moreover, SDS-polyacrylamide gel electrophoresis shows that the toxins of interest represent the majority of the proteins in the bacterial sacculus (FIG. 7 C).

SEQUENCES

SEQ ID NO 1-PpapR Promoter region of the papR gene of Bacillus thuringiensis
CTAGAACCATGAATTAAAAGAATCACTTATAAAAAAAATGAAGAAATAAAAAAGACAT
AAAGAACAAATA
TGCATAATTGCATAAAGTCTGGATAATTTTTCATGATATATTTAAAGAAAAAATGCGG
SEQ ID NO 2-PplcB Promoter region of the plcB gene of Bacillus thuringiensis
AGCATGTGTATGCTTTACTCTTTTTTTACATTAGTTTAGACAAGCCTTAATAATAGTTTAT
TAAAATGAAAGT
GTATTCATTCATTATATTCACTGTGTATAAAGTTATAATGATATGAACATTTGCATATTT
TAATTTAGTGATA
GAAATTTCGTGAAAGGTGGGATATTCTAGTCATAGGTTAACCGGACGACATCATAGGAT
CCTAACAAAATG
TTTACAATAATTCAATTATAAAATGGAGG
SEQ ID NO 3-PnprA Promoter region of the nprA gene of Bacillus thuringiensis
TTTTTTCAATATTTGTTCCTCAAAATTCTACAAAACTTGAGAAATAAATTAATTGAATTT
TTAGTATATTAATA
GTGGAAACATAATGCTAATATGAAACTACTCTTTTTCAAAAAAATTTTTATTAGGGGGA
AGGTTCATG
SEQ ID NO 4-PnprR Promoter region of the nprR gene of Bacillus thuringiensis
GAAGTGAAGTCAAGAGAGAAAAAAATGAAATTATGCATATTTTTTAGAAAATTTATATT
TATCAATTTATAT
TTCTCCGAATTTTATGTATTATTAGAGTAATGGGGTAATGAGAATGGAGG
SEQ ID NO 5-PoppA Promoter region of the oppA gene of Bacillus thuringiensis
CTAGACCGTCAAAATATTACTAGAATTATTATACTATAAAAGCTATAATAAGTACTAGG
ATTAATTTTTTGAA
AATTATACGCAATTAAAGGTCTGATTTTAAGATGATGGTAGCAATGTTAATGTATCCCTT
TACGAAAAATTT
AAAATATAAATATTTTATTAAAAATTTTAACAAAAAAACAAAAAACTATATTCACAATT
GTTATATTATATGT
ATAATGAAAATTGTAGAAACTTGGAGATTATTCTTTCAATTCTACTTTTTAACAAGTGAG
GGTACAGGAAGT
GCAATTAGGGGAGG
SEQ ID NO 6-PppC Promoter region of the nppC gene of Bacillus thuringiensis
ATTCCACTTTTATATAAAAAGATTTTTGAATATTTTTTCAACTTACCCTTGTCAAACATTG
TCAATTTATATTA
AAATAACAACATACAAAATATTTAGTCTTTTCAGAAAAGATGGAGGGATAGCCATG
SEQ ID NO 7-PinhAl Promoter region of the inhA1 gene of Bacillus thuringiensis
AATTGTGATATATTCGTATGCTAACTATGAAATTTTTACAAATATATTAAAAATATTACA
TGATATGACTAAA
TATTGAAAAAATATTGAATTTTTAATAAAATTCAATTTGTAATACATATTATTTATTAGG
GGAGGAAATATG
GGATG
SEQ ID NO 8-PcalY Promoter region of the calY gene of Bacillus thuringiensis.
AAGCAAGACTAGTAATATTTATACGAGTGTGAGGGAACGTTAGCCCTCACCTCTTTGTT
TTTCTTTTTTCTTA
TAGTATTAAATGATTTGAGTGTGAAAAAAGTTATAAATTATCATTGTTTTTTTCTGAAAA
GTCTAAATGATAT
TGAGAAATAAAAATAACTGAAAATATTAATAAATATGTGTTGTGTTTATATAGGGTTGT
TCGTTATAATGAA
CATAAGGTTTTTAAAAAAGAACACATATTAGCTAAGCTAATATAGTTTTCTTTACATCTC
TTATTGAAAAATA
AGTGATAAAAATATAAAAAAAAGCTAGGGGGAATTGATT

The +1 of transcription is indicated in bold. The −10 boxes of the promoters are underlined with a thin line. The −35 boxes are underlined with 2 lines. The DNA sequence recognized by PlcR (PlcR box) of the PpapR and PpIcB promoters is underlined with a thick line.

-STAB-SD Bacillus thuringiensis
SEQ ID NO 9
gaaaggaggg atgcc
-Tcry1Ac-Bacillus thuringiensis
SEQ ID NO 10
aaactcaggt ttaaatatcg ttttcaaatc aattgtccaa gagcagcatt acaaatagat
aagtaatttg ttgtaatgaa aaacggacat cacctccatt gaaacggagt gatgtccgtt
ttactatgtt attttctagt aatacatatg tatagagcaa cttaatcaag cagagatatt
ttcacctatc gatgaaaata tctctgcttt ttcttttttt at
Sequence containing 2 LysM domains as described by Shao et al., 2009, Microb Cell
Fact 8, 48. doi: 10.1186/1475-2859-8-48.
SEQ ID NO 11
ATGATTCAAATTGTAACGGTTCGTAGCGGTGATAGCGTATATAGCTTGGCATCAAAATA
TGGATCAACACC
TGACGAAATAGTAAAAGACAATGGACTAAATCCCGCTGAAACGCTCGTTGTTGGTCAG
GCACTTATCGTTA
ATACGAAAGGAAATAATTATTATGTACAGCCTGGTGACAGCCTCTATCGGATTTCTCAA
ACATATAATGTCC
CCCTCGCTAGTTTAGCTAAAGTTAATAATTTATCTTTAAAATCTATTCTCCATGTCGGAC
AACAATTATATGT ACCAAAAGGCACA
SEQ ID NO 12: Sequence containing 3 SLH domains as described by Fedhila et al.,
2006, Mol. Microbiol. 62: 339-355. doi: 10.1111/j.1365-2958.2006.05362.x.
GAAGATGAGAAAACAGAAGTGGTAGAATTTAAAGATGTACCAAAGGGACATTGGTCAG
AAGAAGCAATT
AATTACTTAGCGAAAGAAAAATTATTTATAGGCTATGGAAATGGTGAATTTGGATTTGG
TGATAACATTACT
CGTGGACAAGTAGCACTTCTAATACAAAGATATTTAAAATTAGAAAATAATCTAGAACA
AAAAACGGCATT
TACAGATACGAAAGGAAATATGTATGAAACGGCTATTGATGCAGTGGTTCAAGCTGGA
ATTATGACAGGC
TATGGAAATGGTATGTTCCGTCCGGATGGAGTATTAACTCGATATGAAATGTCAGTAGT
ACTACAAAGAGT
ATTTCAGTTAAAAGAAAATGAAAATAGTGCAGAGAATTTTAAAGATGTACCAAATGGC
CATTGGGCGAAA
GGATATGTGAAAGCTTTAGTGGATAATAAAATATCAAAAGGCGACGGGGAAGGGAATT
TTTTAGGAGATA
ATTTCGTAACACGTGAACAATACGCACAGTTTTTGTATAATGCAATAAAGAAA

1. A non-sporulating bacterial strain of the genus Bacillus containing a recombinant plasmid comprising an expression cassette composed of:
(i) a strong promoter active in a stationary phase and regulated by a regulator chosen from CodY, AbrB, SinR, PlcR and NprR; and
(ii) a sequence of a gene encoding a protein of interest.

2. The bacterial strain according to claim 1, wherein the strong promoter is chosen from PoppA, PnppC, PinhA1, PcalY, PpapR, PpIcB, PnprR and PnprA, preferably, the strong promoter is chosen from PoppA or PnppC.

3. The bacterial strain according to claim 1, wherein the sporulation gene chosen from Spo0A and sigE is inactivated by interruption or modification of the sequence or by deletion of all or part of the gene.

4. The bacterial strain according to claim 3, wherein:
(i) the strain is mutated in the Spo0A gene; and
(ii) the strong promoter is chosen from PpapR, PnprA, PnprR, PpIcB, PoppA and PnppC.

5. The bacterial strain according to claim 1, wherein:
(i) the strain is mutated in a sigE gene; and
(ii) the strong promoter is chosen from PinhA1 and PcalY.

6. The bacterial strain according to claim 1, wherein the expression cassette further comprises a stabilizing sequence of mRNA and/or a terminating sequence of a cry1Ac gene.

7. The bacterial strain according to claim 6, wherein the stabilizing sequence of mRNA is a sequence STAB-SD.

8. The bacterial strain according to claim 1, wherein the plasmid is a plasmid chosen from pHT304, pHT315 and pHT370 pHT73, pBC16, pE194, pC194 and pBM299.

9. The bacterial strain according to claim 1, wherein the expression cassette further comprises the sequences encoding for an anchor or export sequence of proteins.

10. The bacterial strain according to claim 1, wherein the bacterial strain is a strain of Bacillus thuringiensis.

11. A method for producing a protein of interest comprising the steps of:
a—preparing the bacterial strain according to claim 1;
b—culturing the bacterial strain in the stationary phase; and
c—optionally, purifying the protein of interest.

12. A method for producing a protein of interest comprising the steps of:
a—preparing the bacterial strain according to claim 2;
b—culturing the bacterial strain in the stationary phase; and
c—optionally, purifying the protein of interest.

13. A method for producing a protein of interest comprising the steps of:
a—preparing the bacterial strain according to claim 3;
b—culturing the bacterial strain in the stationary phase; and
c—optionally, purifying the protein of interest.

14. A method for producing a protein of interest comprising the steps of:
a—preparing the bacterial strain according to claim 4;
b—culturing the bacterial strain in the stationary phase; and
c—optionally, purifying the protein of interest.

15. A method for producing a protein of interest comprising the steps of:
a—preparing the bacterial strain according to claim 5;
b—culturing the bacterial strain in the stationary phase; and
c—optionally, purifying the protein of interest.

16. The bacterial strain according to claim 2, wherein the sporulation gene chosen from Spo0A and sigE is inactivated by interruption or modification of the sequence or by deletion of all or part of the gene.

17. The bacterial strain according to claim 1, wherein:
(iii) the strain is mutated in a Spo0A gene; and
(iv) the strong promoter is chosen from PpapR, PnprA, PnprR, PpIcB, PoppA and PnppC.

18. The bacterial strain according to claim 2, wherein:
(v) the strain is mutated in a Spo0A gene; and
(vi) the strong promoter is chosen from PpapR, PnprA, PnprR, PpIcB, PoppA and PnppC.

19. The bacterial strain according to claim 2, wherein:
(iii) the strain is mutated in a sigE gene; and
(iv) the strong promoter is chosen from PinhA1 and PcalY.

20. The bacterial strain according to claim 3, wherein:
(v) the strain is mutated in the sigE gene; and
(vi) the strong promoter is chosen from PinhA1 and PcalY.
